(19) United States
(12) Patent Application Publication
(10) Pub. No.: US 2021/0237002 A1
(43) Pub. Date: Aug. 5, 2021

(54) RAPID AND FACILE MEMBRANE ADSORBER FABRICATION WITH ULTRA HIGH BINDING CAPACITY

(71) Applicant: RENSSELAER POLYTECHNIC INSTITUTE, Troy, NY (US)

(72) Inventors: Somdatta Bhattacharya, Troy, NY (US); John Joseph Keating, Troy, NY (US); Georges Belfort, Slingerlands, NY (US)

(73) Assignee: RENSSELAER POLYTECHNIC INSTITUTE, Troy, NY (US)

(21) Appl. No.: 16/972,044

(22) PCT Filed: Jun. 5, 2019
(86) PCT No.: PCT/US19/35551

§ 371 Date: Dec. 4, 2020

Related U.S. Application Data
(60) Provisional application No. 62/680,743, filed on Jun. 5, 2018.

(51) Int. Cl. B01D 69/14; C08J 7/16; G01N 33/543; B01D 69/02; B01D 67/00; B01D 71/82

(52) U.S. Cl. CPC B01D 69/147; C08J 7/16; G01N 33/54353; B01D 69/02; B01D 2325/12; B01D 71/82; C08J 2300/202; B01D 2323/38; B01D 67/0093

(57) ABSTRACT

Functionalized membranes are produced via grafting of polymer brushes to the membrane surface for use, e.g., in separation and purification of biomolecules. One or more initiators are attached to the membrane surface. A reactant substrate, such as a copper metal plate, is placed adjacent the membrane. A reaction medium is then provided in fluid contact with the membrane and the reactant substrate, the reaction medium including one or more monomers, one or more ligands, and one or more solvents. The polymer brushes are grown on the membrane via Cu(0)-mediated controlled radical polymerization involving the reactant substrate and the reaction medium. This reaction process uses fewer numbers and amounts of chemicals compared to other controlled radical polymerization reactions such as ATRP. The reaction can take place at room temperature, which is more energy efficient than other CRPs which occur at a much higher temperatures. The reaction process described herein is also sixteen times faster than the standard ATRP method without sacrificing subsequent separation performance.

### CROSS REFERENCE TO RELATED APPLICATION(S)

This application claims the benefit of U.S. Provisional Application No. 62/680,743, filed Jun. 5, 2018, which is incorporated by reference as if disclosed herein in its entirety.

### BACKGROUND

There is a continual need for improved systems and techniques to facilitate more effective separation of biological material such as proteins, viruses, endotoxins, ribonucleic acids, etc. One particular focus of development in this area has been the controlled growth of polymer brushes to functionalize surfaces of a separation medium such as a membrane. The functionalized surfaces can be tailored to the species being separated to provide fine separation between otherwise stubborn mixtures of species.

Amongst the separation media benefitting from the development of more sophisticated polymer brush growth techniques are charged ion exchange membranes. These membranes can contain either positively functionalized brushes (anion exchange) or negatively functionalized brushes (cation exchange). The brushes then interact with charged molecules primarily through electrostatic interactions to facilitate the desired separation.

Industry and other academic groups have increased performance (binding kinetics and amount bound) of these types of membranes. However, in addition to improved performance, there is a desire for more simplified functionalization processes that still enable high dynamic binding capacity for target species adsorption.

### SUMMARY

Some embodiments of the present disclosure are directed to a method of modifying a membrane including providing a membrane to be modified, the membrane having a surface, attaching one or more initiators to the membrane surface, positioning a reactant substrate adjacent the membrane, providing a reaction medium in fluid contact with the membrane and the reactant substrate, the reaction medium including one or more monomers, one or more ligands, and one or more solvents, and polymerizing a plurality of polymer brushes on the membrane surface. In some embodiments, polymerizing the plurality of polymer brushes on the surface of the membrane is performed at ambient temperature. In some embodiments, the polymerization reaction is quenched after about 25 minutes to about 35 minutes. In some embodiments, the polymerization reaction is quenched after about 30 minutes.

In some embodiments, the one or more initiators include 2-bromoisobutyryl bromide, alkyl chlorides, methyl 2-chloropropionate (MCP), chloroform (CHCl₃), lactose-based octa-functional initiator, or combinations thereof In some embodiments, the one or more monomers include vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butylaminoethyl acrylate, tertiary-butylaminoethyl methacrylate, dimethylaminopropylacrylamide, sulfopropyl methacrylate potassium salt, carboxyethyl acrylate, lauryl methacrylate, poly(ethylene glycol) methacrylate, isobutyl methacrylate, trifluoroethyl methacrylate, poly(propylene) glycol, or combinations thereof In some embodiments, the one or more ligands include pentamethyldiethylenetriamine (PMDETA), tris(2-aminoethyl)amine (Tren), hexamethyltriethylenetetramines (HMTETA), bipyridines (Bipy), 4,4-dinonyl-2,2-bipyridine (diNbpy), diethylenetriamine, or combinations thereof In some embodiments, the one or more solvents include methanol, water, dimethylsulfoxide, dimethylformamide, acetonitrile, or combinations thereof.

In some embodiments, the reactant substrate is positioned about 0.25 mm to about 0.75 mm from the membrane surface. In some embodiments, the reactant substrate is positioned about 0.5 mm from the membrane surface. In some embodiments, the reactant substrate is positioned above the membrane surface on one or more shims.

In some embodiments, the reactant substrate includes copper. In some embodiments, the reactant substrate includes a surface composed of copper metal, and the copper metal surface is positioned facing the membrane surface.

Some embodiments of the present disclosure are directed to a method of modifying a membrane including providing a membrane to be modified, the membrane having a surface. In some embodiments, the method includes attaching one or more initiators to the membrane surface. In some embodiments, the method includes positioning a copper metal plate to provide a gap between the membrane surface and a surface of the copper metal plate. In some embodiments, the method includes providing a reaction medium to the gap, the reaction medium including one or more monomers, one or more ligands, and one or more solvents. In some embodiments, the method includes polymerizing a plurality of polymer brushes on the membrane surface at ambient temperature.

Some embodiments of the present disclosure are directed to a modified membrane including a porous substrate layer and an active layer positioned on the substrate layer, the active layer including a plurality of polymer brushes. In some embodiments, the plurality of polymer brushes are grown on the substrate layer via Cu(0)-mediated controlled radical polymerization. In some embodiments, the plurality of polymer brushes are individually positively charged, negatively charged, apolar, or combinations thereof In some embodiments, the plurality of polymer brushes are composed of vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butylaminoethyl acrylate, tertiary-butylaminoethyl methacrylate, dimethylaminopropylacrylamide, sulfopropyl methacrylate potassium salt, carboxyethyl acrylate, lauryl methacrylate, poly(ethylene glycol) methacrylate, isobutyl methacrylate, trifluoroethyl methacrylate, poly(propylene) glycol, or combinations thereof.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic drawing of a modified membrane according to some embodiments of the present disclosure;

FIG. 2 is a chart of a method for Cu(0)-mediated controlled radical polymerization according to some embodiments of the present disclosure;

FIG. 3 is a chart of a method of modifying a substrate according to some embodiments of the present disclosure;

FIG. 4 is a schematic drawing of a process of modifying a substrate according to some embodiments of the present disclosure; and

FIG. 5 is a graph comparing the dynamic binding capacity of modified membranes according to some embodiments of the present disclosure to membranes described in the literature.

### DETAILED DESCRIPTION

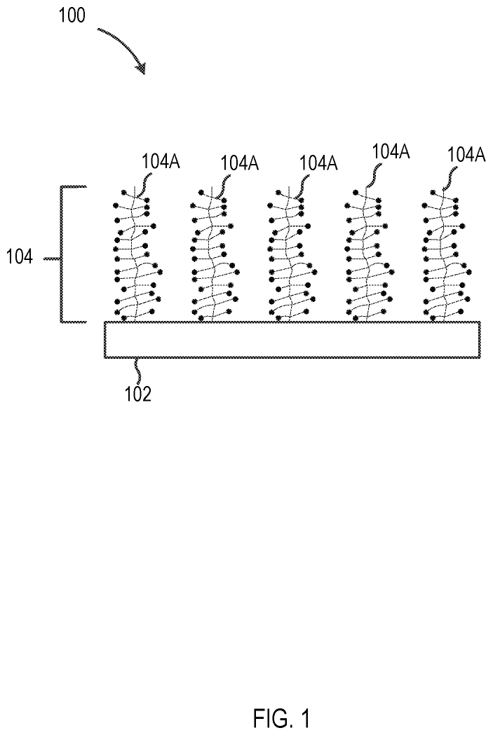

Referring now to FIG. 1, some aspects of the disclosed subject matter include a modified membrane 100 for use, e.g., in the separation of host cell proteins, nucleic acids, viruses, virus-like particles, endotoxins, leached ligands, etc., or combinations thereof In some embodiments, membrane 100 includes a porous substrate layer 102. In some embodiments, porous substrate layer 102 is composed of any material or combination of materials suitable for facilitating the desired separation, e.g., cellulose, polyethersulfone, poly(aryl sulfone), polyimide, cellulose acetate, polypropylene, polyethylene, etc., or combinations thereof.

In some embodiments, an active layer 104 is positioned on the substrate layer 102. Active layer 104 includes a plurality of polymer brushes 104A that are composed of a plurality of monomers. In some embodiments, polymer brushes 104A are branched, unbranched, or combinations thereof. In some embodiments, polymer brushes 104A are positively charged, negatively charged, apolar, or combinations thereof In some embodiments, polymer brushes 104A are individually composed of monomers including vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butylaminoethyl acrylate, tertiary-butylaminoethyl methacrylate, dimethylaminopropylacrylamide, sulfopropyl methacrylate potassium salt, carboxyethyl acrylate, lauryl methacrylate, poly(ethylene glycol) methacrylate, isobutyl methacrylate, trifluoroethyl methacrylate, poly(propylene) glycol, other vinyl-containing monomers, or combinations thereof.

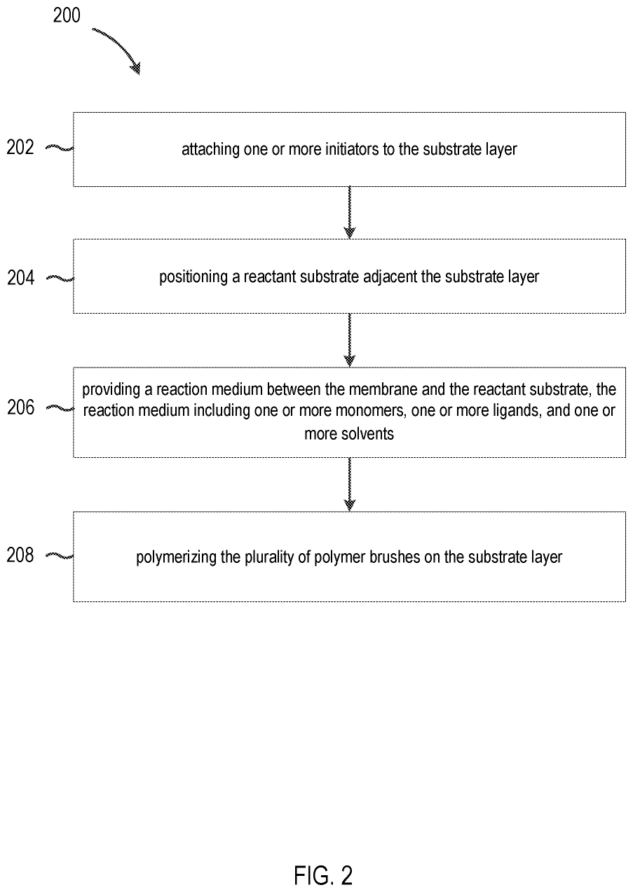

Referring now to FIG. 2, in some embodiments, polymer brushes 104A are grown on substrate layer 102 via Cu(0)-mediated controlled radical polymerization 200. In some embodiments, at 202, one or more initiators are attached to the substrate layer. At 204, a reactant substrate is positioned adjacent the substrate layer, as will be discussed in greater detail below. At 206, a reaction medium is provided between the membrane and the reactant substrate, the reaction medium including one or more monomers, one or more ligands, and one or more solvents, as will also be discussed in greater detail below. At 208, polymer brushes are polymerized on the substrate layer. In some embodiments, polymerizing 208 the polymer brushes on the membrane surface is performed at ambient temperature.

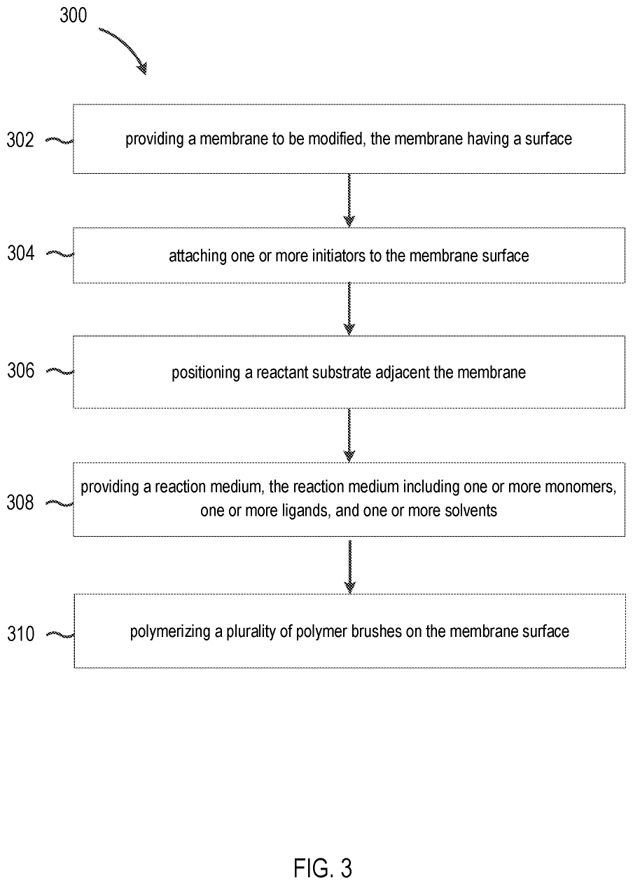

Referring now to FIG. 3, some embodiments of the present disclosure are directed to a method 300 of modifying a substrate. In some embodiments, at 302, the substrate to be modified is provided, the substrate having a surface. In some embodiments, the substrate is composed of any suitable material so long as it is capable of being surface-functionalized with one or more initiators for initiating formation of polymer brushes on the surface, as will be discussed in greater detail below. In some embodiments, the substrate is a membrane, e.g., membrane 100 described above. As used herein, the term “surface” should be understood to include an outer boundary of the substrate, as well as extending at least partially into pores or cavities in the substrate, e.g., in a separation membrane. At 304, one or more initiators are attached to the substrate surface, e.g., at a location on the substrate where modification is desired. In some embodiments, the one or more initiators include 2-bromoisobutyryl bromide, alkyl chlorides, methyl 2-chloropropionate (MCP), chloroform (CHCl₃), lactose-based octa-functional initiator, or combinations thereof. At 306, a reactant substrate is positioned adjacent the substrate, e.g., the substrate surface, at a location on the substrate where modification is desired, etc., or combinations thereof In some embodiments, material of the reaction substrate is utilized as a mediator in the polymerization of polymer brushes at the one or more initiators, as will be described in greater detail below. In some embodiments, the reactant substrate includes copper. In some embodiments, the reactant substrate includes a surface composed of copper metal, and the copper metal surface is positioned facing the substrate surface. In some embodiments, the reactant substrate is or includes a copper metal plate. In some embodiments, the reaction substrate is positioned at a distance from the substrate surface to be suitable as a mediator in the polymerization of polymer brushes at the one or more initiators. In some embodiments, the reactant substrate is positioned about 0.25 mm to about 0.75 mm from the substrate surface. In some embodiments, the reactant substrate is positioned about 0.5 mm from the substrate surface.

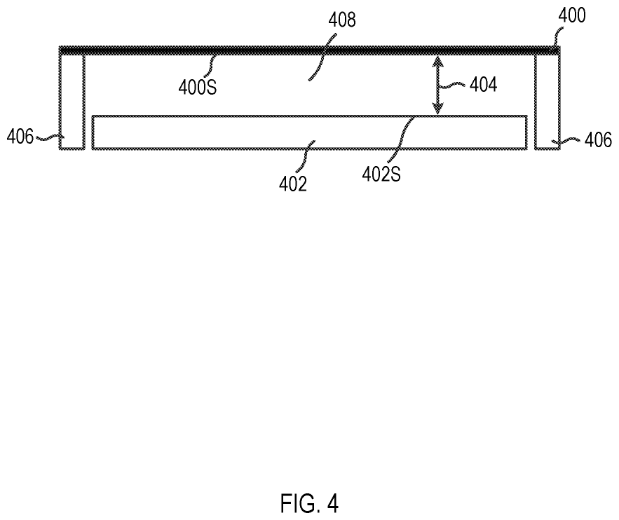

Referring now to FIG. 4, in some embodiments of the present disclosure, a reactant substrate 400 is positioned adjacent a substrate to be modified 402 to provide a gap 404 between the substrate surface 402S and the reactant substrate surface 400S. In some embodiments, reactant substrate 400 is positioned above the substrate surface 402S on one or more shims 406. In some embodiments, shim 406 is shaped to position reactant substrate surface 400S in the desired proximity to the substrate surface 402S, yet still allow the presence of a reaction medium 408 in gap 404, as will be discussed in greater detail below. In some embodiments, shim 406 is composed of a suitable inert material so as to not interfere in the formation of polymer brushes on substrate surface 402S.

Referring again to FIG. 3, at 308, a reaction medium is provided in fluid contact with the substrate and the reactant substrate. In some embodiments, the reaction medium is provided to a gap between the substrate and the reaction substrate. In some embodiments, the reaction medium includes one or more monomers, one or more ligands, and one or more solvents. In some embodiments, the one or more monomers include vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butylaminoethyl acrylate, tertiary-butylaminoethyl methacrylate, dimethylaminopropylacrylamide, sulfopropyl methacrylate potassium salt, carboxyethyl acrylate, lauryl methacrylate, poly(ethylene glycol) methacrylate, isobutyl methacrylate, trifluoroethyl methacrylate, poly(propylene) glycol, other vinyl-containing monomers, or combinations thereof. In some embodiments, the one or more ligands include pentamethyldiethylenetriamine (PMDETA), tris(2-aminoethyl)amine (Tren), hexamethyltriethylenetetramines (HMTETA), bipyridines (Bipy), 4,4-dinonyl-2,2-bipyridine (diNbpy), diethylenetriamine, or combinations thereof In some embodiments, the one or more solvents include methanol, water, dimethylsulfoxide, dimethylformamide, acetonitrile, or combinations thereof. In some embodiments, the solvent includes methanol and water in about a 1:1 v/v ratio to about a about a 1:3 v/v ratio. In some embodiments, the solvent includes methanol and water in about a 1:2 v/v ratio. At 310, a plurality of polymer brushes are polymerized on the substrate, e.g., the substrate surface. As discussed above, in some embodiments, polymer brushes are branched, unbranched, or combinations thereof. In some embodiments, polymer brushes are positively charged, negatively charged, apolar, or combinations thereof In some embodiments, material of the reactant substrate is utilized in polymerization of the polymer brushes. In some embodiments, the polymerization reaction of step 310 is quenched after about 25 minutes to about 35 minutes. In some embodiments, the polymerization reaction of step 310 is quenched after about 30 minutes. In some embodiments, the polymerization reaction of step 310 is performed at ambient temperature.

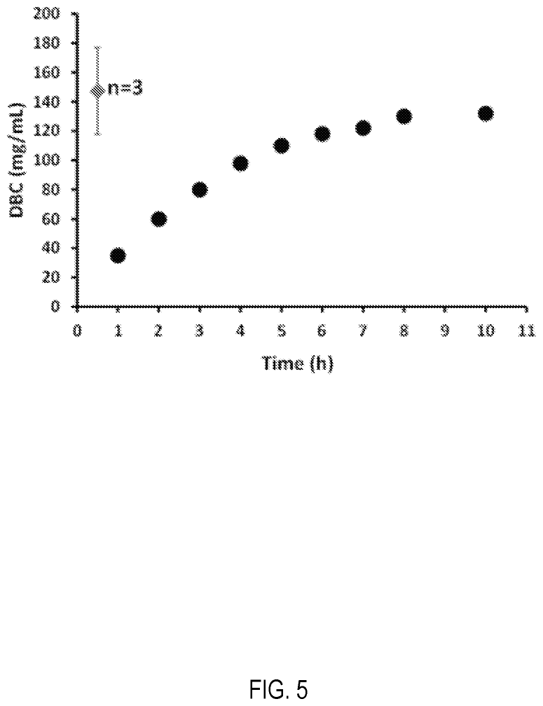

By way of example, and now referring to FIG. 5, the dynamic binding capacity of modified membranes consistent with the present disclosure were compared with high performing adsorption membranes from the literature. As shown in FIG. 5, the modified membranes consistent with the present disclosure showed drastically improved performance (diamonds) compared to the literature (circles). Further, the polymerization time used in making membranes of the present disclosure was 1/16^{th} that of the literature, and the membranes of the present disclosure were also modified at room temperature, demonstrating how the faster membrane modification methods of the present disclosure are also more energy efficient.

Systems and methods of the present disclosure are advantageous to functionalize substrates by grafting polymer brushes onto surfaces thereof, e.g., to create ion-exchange membranes. Firstly, the systems and methods of the present disclosure are simpler and have increased ease-of use compared to those of the prior art. The systems and methods use fewer numbers and amounts of chemicals compared to other controlled radical polymerization reactions such as atom transfer radical polymerization. The polymer brush polymerization reactions of the present disclosure can be conducted with only solvent, monomer and ligand in the presence of a copper plate, i.e., without the presence of copper salts and reducing agents. The reaction volumes can be limited to a few milliliters, yet still yield dense polymer chains in a short period. This minimal volume requirement is advantageous while scaling up the polymer brush coatings to entire sheets of substrate rolls, saving process time and reducing footprint of chemicals used to manufacture the membranes. Further, the polymer brush polymerization reaction can take place at room temperature, which gives an added benefit compared with other CRPs which occur at a much higher temperature. This lower temperature means the methods of the present disclosure are more energy efficient than those of the prior art. The systems and methods of the present disclosure are also faster than alternative methods using ATRP, in fact up to at least sixteen times faster than the highest binding capacity membranes reported in the literature and at room temperature rather than 60° C. for 8 hr. for the standard ATRP method. Finally, membranes prepared using systems and methods of the present disclosure are advantageously used in separation and purification of biomolecules like host cell proteins, nucleic acids, virus like particles or virus, endotoxins and leached ligands.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A modified membrane, comprising:
a porous substrate layer; and
an active layer positioned on the substrate layer, the active layer including a plurality of polymer brushes;
wherein the plurality of polymer brushes are grown on the substrate layer via Cu(0)-mediated controlled radical polymerization.

2. The membrane according to claim 1, wherein the plurality of polymer brushes are individually positively charged, negatively charged, apolar, or combinations thereof.

3. The membrane according to claim 2, wherein the plurality of polymer brushes are composed of vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butylaminoethyl acrylate, tertiary-butylaminoethyl methacrylate, dimethylaminopropylacrylamide, sulfopropyl methacrylate potassium salt, carboxyethyl acrylate, lauryl methacrylate, poly(ethylene glycol) methacrylate, isobutyl methacrylate, trifluoroethyl methacrylate, poly(propylene) glycol, or combinations thereof.

4. The membrane according to claim 1, wherein the Cu(0)-mediated controlled radical polymerization includes:
attaching one or more initiators to the substrate layer;
positioning a reactant substrate adjacent the substrate layer;
providing a reaction medium between the membrane and the reactant substrate, the reaction medium including one or more monomers, one or more ligands, and one or more solvents; and
polymerizing the plurality of polymer brushes on the substrate layer.

5. The membrane according to claim 4, wherein the reactant substrate includes a copper metal plate.

6. The membrane according to claim 1, wherein polymerizing the plurality of polymer brushes on the membrane surface is performed at ambient temperature.

7. A method of modifying a membrane, comprising:
providing a membrane to be modified, the membrane having a surface;
attaching one or more initiators to the membrane surface;
positioning a reactant substrate adjacent the membrane;
providing a reaction medium in fluid contact with the membrane and the reactant substrate, the reaction medium including one or more monomers, one or more ligands, and one or more solvents; and
polymerizing a plurality of polymer brushes on the membrane surface.

8. The method according to claim 7, wherein the reactant substrate is positioned about 0.25 mm to about 0.75 mm from the membrane surface.

9. The method according to claim 7, wherein the reactant substrate is positioned above the membrane surface on one or more shims.

10. The method according to claim 7, wherein the one or more initiators include 2-bromoisobutyryl bromide, alkyl chlorides, methyl 2-chloropropionate (MCP), chloroform (CHCl₃), lactose-based octa-functional initiator, or combinations thereof.

11. The method according to claim 7, wherein the one or more monomers include vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butyl aminoethyl acrylate, tertiary-butylaminoethyl methacrylate, dimethylaminopropylacrylamide, sulfopropyl methacrylate potassium salt, carboxyethyl acrylate, lauryl methacrylate, poly(ethylene glycol) methacrylate, isobutyl methacrylate, trifluoroethyl methacrylate, poly(propylene) glycol, or combinations thereof.

12. The method according to claim 7, wherein the one or more ligands include pentamethyldiethylenetriamine (PMDETA), tris(2-aminoethyl)amine (Tren), hexamethyltriethylenetetramines (HMTETA), bipyridines (Bipy), 4,4-dinonyl-2,2-bipyridine (diNbpy), diethylenetriamine, or combinations thereof.

13. The method according to claim 7, wherein the one or more solvents include methanol, water, dimethylsulfoxide, dimethylformamide, acetonitrile, or combinations thereof.

14. The method according to claim 7, wherein the reactant substrate includes copper.

15. The method according to claim 14, wherein the reactant substrate includes a surface composed of copper metal, and the copper metal surface is positioned facing the membrane surface.

16. The method according to claim 7, wherein polymerizing the plurality of polymer brushes on the surface of the membrane is performed at ambient temperature.

17. The method according to claim 7, wherein the polymerization reaction is quenched after about 25 minutes to about 35 minutes.

18. A method of modifying a membrane, comprising:
providing a membrane to be modified, the membrane having a surface;
attaching one or more initiators to the membrane surface;
positioning a copper metal plate to provide a gap between the membrane surface and a surface of the copper metal plate;
providing a reaction medium to the gap, the reaction medium including one or more monomers, one or more ligands, and one or more solvents; and
polymerizing a plurality of polymer brushes on the membrane surface at ambient temperature,
wherein the polymerization reaction is quenched after about 30 minutes.

19. The method according to claim 18, wherein
the one or more initiators include vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butylaminoethyl acrylate, tertiary-butylaminoethyl methacrylate, dimethylaminopropylacrylamide, sulfopropyl methacrylate potassium salt, carboxyethyl acrylate, lauryl methacrylate, poly(ethylene glycol) methacrylate, isobutyl methacrylate, trifluoroethyl methacrylate, poly(propylene) glycol, or combinations thereof;
the one or more ligands include pentamethyldiethylenetriamine (PMDETA), tris(2-aminoethyl)amine (Tren), hexamethyltriethylenetetramines (HMTETA), bipyridines (Bipy), 4,4-dinonyl-2,2-bipyridine (diNbpy), diethylenetriamine, or combinations thereof; and
the one or more solvents include methanol, water, dimethylsulfoxide, dimethylformamide, acetonitrile, or combinations thereof.

20. The method according to claim 18, wherein the gap is about 0.5 mm.
